(19) United States
(12) Patent Application Publication
(10) Pub. No.: US 2022/0227722 A1
(43) Pub. Date: Jul. 21, 2022

(54) SPHINGOSINE 1 PHOSPHATE RECEPTOR MODULATOR

(71) Applicant: RECEPTOS LLC, New York, NY (US)

(72) Inventors: Philip TURNBULL, Summit, NJ (US); Esther MARTINBOROUGH, Summit, NJ (US); Maurice MARSINI, Summit, NJ (US)

(21) Appl. No.: 17/606,725

(22) PCT Filed: Mar. 27, 2020
(86) PCT No.: PCT/US2020/025142

§ 371 Date: Oct. 26, 2021

Related U.S. Application Data
(60) Provisional application No. 62/839,495, filed on Apr. 26, 2019.

(51) Int. Cl. C07D 271/06

(52) U.S. Cl. CPC C07D 271/06

(57) ABSTRACT

A compound is provided having following structure Compound (1): or a pharmaceutically acceptable salt, homolog, hydrate or solvate thereof. Also provided is a pharmaceutical composition comprising Compound (1) or a pharmaceutically acceptable salt, homolog, hydrate or solvate thereof, in combination with a pharmaceutically acceptable carrier or diluent.

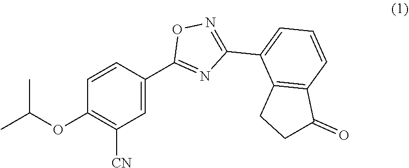 [SMILES: CC(C)Oc1ccc(-c2nc(-c3cccc4c3CCC4=O)no2)cc1C#N]

### BACKGROUND

### Technical Field

A modulator of the sphingosine-1-phosphate receptor is provided for treatment of a malcondition for which activation of the same is medically indicated.

### Description of the Related Art

The S1P₁/EDG₁ receptor is a G-protein coupled receptor (GPCR) and is a member of the endothelial cell differentiation gene (EDG) receptor family. Endogenous ligands for EDG receptors include lysophospholipids, such as sphingosine-1-phosphate (S1P). Like all GPCRs, ligation of the receptor propagates second messenger signals via activation of G-proteins (alpha, beta and gamma). Development of small molecule S1P₁ agonists and antagonists has provided insight into some physiological roles of the S1P₁/S1P-receptor signaling system. To this end, S1P receptors are divided into five subtypes (i.e., S1P₁, S1P₂, S1P₃, S1P₄, and S1P₅), which subtypes are expressed in a wide variety of tissues and exhibit different cell specificity. Agonism of the S1P₁ receptor perturbs lymphocyte trafficking, sequestering them in lymph nodes and other secondary lymphoid tissue. This leads to rapid and reversible lymphopenia, and is probably due to receptor ligation on both lymphatic endothelial cells and lymphocytes themselves (Rosen et al, Immunol. Rev., 195:160-177, 2003).

### BRIEF SUMMARY

In brief, a modulator of the sphingosine-1-phosphate receptor is provided for treatment of a malcondition for which activation of the same is medically indicated.

In one embodiment, a compound is provided in an isolated or purified form having the following structure (also referred to as “Compound 1” or “Cpd 1”):

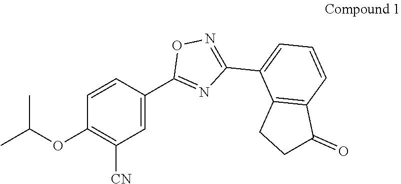 [SMILES: CC(C)Oc1ccc(-c2nc(-c3cccc4c3CCC4=O)no2)cc1C#N]

or a pharmaceutically acceptable salt, homolog, hydrate or solvate thereof.

In another embodiment, a pharmaceutical composition is provided comprising Compound 1 or a pharmaceutically acceptable salt, homolog, hydrate or solvate thereof, in combination with a pharmaceutically acceptable carrier or diluent.

### BRIEF DESCRIPTION OF THE DRAWING

FIG. 1 shows the mean plasma concentration relative to lymphocyte count (CD4⁺, 8⁺ and B220⁺) for Compound 1.

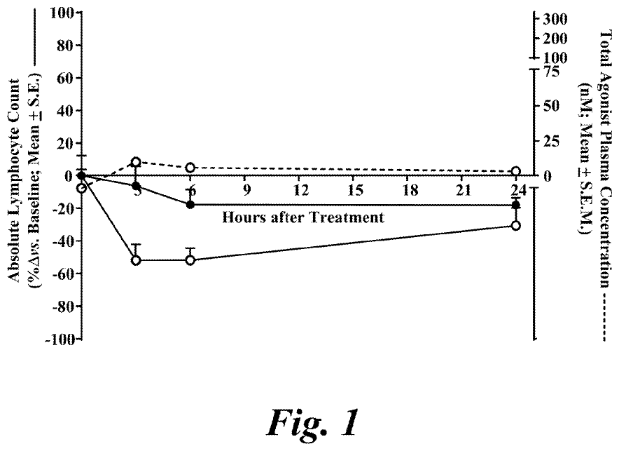

### DETAILED DESCRIPTION OF THE DISCLOSURE

As used in the specification and the appended claims, the singular forms “a,” “an” and “the” include plural referents unless the context clearly dictates otherwise. Further, the words “comprising,” “including” and “having” are open-ended terms as used herein, and do not preclude the existence of additional elements or components.

The present invention is directed to a compound and composition which modulates an S1P receptor. S1P receptors are divided into five subtypes (i.e., S1P₁, S1P₂, S1P₃, S1P₄ and S1P₅), which subtypes are expressed in a wide variety of tissues and exhibit different cell specificity. The compound disclosed herein modulates one or more of these subtypes. In one embodiment, the compound is an “S1P₁” modulator as it modulates subtype 1 of a sphingosine-1-phosphate receptor. In another embodiment, the compound modulates subtype 1 and another subtype, such as subtype 5. As used herein, an “S1P₁ modulator” is understood to encompass a compound that modulates the S1P₁ subtype alone, or modulates the S1P₁ subtype as well as one or more other subtypes. In one embodiment, the S1P₁ modulator modulates both the S1P₁ subtype and the S1P₅ subtype.

As used herein, a “modulator” of the S1P₁ receptor is a compound which, when administered to a subject, provides the desired interaction with the target receptor, either by way of the compound acting directly on the receptor itself, or by way of a metabolite of the compound acting on the receptor. Upon administration to a subject, the compound of this invention modulates the S1P₁ receptor by activating on the receptor for signal transduction. Such compound is also referred to herein as an “agonist” or “S1P₁ agonist”. Such an S1P₁ agonist can be selective for action on S1P₁. For example, the compound may be selective for action on S1P₁ at a lower concentration than on other subtypes of the S1P receptor family.

In one embodiment, a compound is provided in an isolated or purified form having the following structure (“Compound 1”):

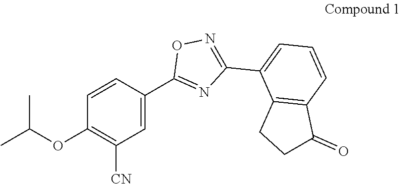 [SMILES: CC(C)Oc1ccc(-c2nc(-c3cccc4c3CCC4=O)no2)cc1C#N]

or a pharmaceutically acceptable salt, homolog, hydrate or solvate thereof. In more specific embodiments, the compound is provided having a purity in excess of 90% (w/w), having a purity in excess of 95% (w/w), or having a purity in excess of 98% (w/w). In a further embodiment, the compound is provided having a purity in excess of 99% (w/w).

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium and alkyl ammonium salts such as tromethamine salts, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present disclosure may take the form of salts. The term “salts” embraces addition salts of free acids or free bases which are compounds of the disclosure. Salts can be “pharmaceutically-acceptable salts.” The term “pharmaceutically acceptable salt” refers to salts which possess toxicity profiles within a range that affords utility in pharmaceutical applications. Pharmaceutically unacceptable salts may nonetheless possess properties such as high crystallinity, which have utility in the practice of the present disclosure, such as for example utility in process of synthesis, purification or formulation of compounds of the disclosure.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4 hydroxybenzoic, phenylacetic, mandelic, embonic (pamoic), methanesulfonic, ethanesulfonic, benzenesulfonic, pantothenic, trifluoromethanesulfonic, 2 hydroxyethanesulfonic, p toluenesulfonic, sulfanilic, cyclohexylaminosulfonic, stearic, alginic, β hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds of the disclosure include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′ dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of compounds, for example in their purification by recrystallization. All of these salts may be prepared by conventional means from the corresponding compound by reacting, for example, the appropriate acid or base with the compound. The term “pharmaceutically acceptable salts” refers to nontoxic inorganic or organic acid and/or base addition salts, see, for example, Gould et al., Salt Selection for Basic Drugs (1986), Int J. Pharm., 33, 201-217, incorporated by reference herein.

Non-limiting examples of potential salts of this disclosure include but are not limited to hydrochloride, citrate, glycolate, fumarate, malate, tartrate, mesylate, esylate, cinnamate, isethionate, sulfate, phosphate, diphosphate, nitrate, hydrobromide, hydroiodide, succinate, formate, acetate, dichloroacetate, lactate, p-toluenesulfonate, pamitate, pidolate, pamoate, salicylate, 4-aminosalicylate, benzoate, 4-acetamido benzoate, glutamate, aspartate, glycolate, adipate, alginate, ascorbate, besylate, camphorate, camphorsulfonate, camsylate, caprate, caproate, cyclamate, laurylsulfate, edisylate, gentisate, galactarate, gluceptate, gluconate, glucuronate, oxoglutarate, hippurate, lactobionate, malonate, maleate, mandalate, napsylate, napadisylate, oxalate, oleate, sebacate, stearate, succinate, thiocyanate, undecylenate, and xinafoate.

A “homolog” of a compound of the disclosure is a compound having one or more atoms of the compound replaced by an isotope of such atom. For example, homologs include compounds with deuterium in place of one or more hydrogen atoms of the compound such as compounds of the disclosure in which the methyl groups of the isopropoxy moiety of Formulas I-R and I-S are fully or partially deuterated (e.g., (D₃C)₂CHO—). Isotopic substitutions which may be made in the formation of homologs of the disclosure include non-radioactive (stable) atoms such as deuterium and carbon 13, as well as radioactive (unstable) atoms such as tritium, carbon 14, iodine 123, iodine 125, and the like.

A “hydrate” is a compound that exists in a composition with water molecules. The composition can include water in stoichiometric quantities, such as a monohydrate or a dihydrate, or can include water in random amounts. As the term is used herein a “hydrate” refers to a solid form, i.e., a compound in water solution, while it may be hydrated, is not a hydrate as the term is used herein.

A “solvate” is a similar composition except that a solvent other that water replaces the water. For example, methanol or ethanol can form an “alcoholate”, which can again be stoichiometric or non-stoichiometric. As the term is used herein a “solvate” refers to a solid form, i.e., a compound in solution in a solvent, while it may be solvated, is not a solvate as the term is used herein.

In another embodiment, a pharmaceutical composition is provided comprising a compound having the following structure (Compound 1):

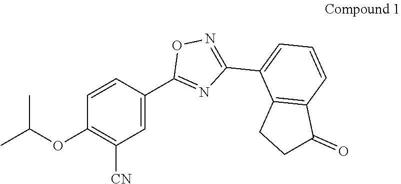 [SMILES: CC(C)Oc1ccc(-c2nc(-c3cccc4c3CCC4=O)no2)cc1C#N]

or a pharmaceutically acceptable salt, homolog, hydrate or solvate thereof, in combination with a pharmaceutically acceptable carrier or diluent. In this embodiment, the terms “pharmaceutically acceptable”, “salt”, “homolog”, “hydrate” and “solvate” are as defined above. With regard to pharmaceutically acceptable carriers or diluents, pharmaceutical compositions may take a variety of different forms, including (but not limited to) forms suitable for oral or intravenous administration. Pharmaceutically acceptable carriers and excipients are known in the art, such as those disclosed in Remington: The Science and Practice of Pharmacy, 22^{nd} Edition, Allen, Lloyd V., Jr. Ed. (2012) (incorporated herein by reference).

Compound 1 can be prepared by techniques known to one skilled in the art, as well as by the procedures disclosed in the following Examples.

### EXAMPLES

### General Methods of Synthesis

¹H NMR (400 MHz) and ¹³C NMR (100 MHz) were obtained in solution of deuteriochloroform (CDCl₃), deuteriomethanol (CD₃OD) or dimethyl sulfoxide—D₆ (DMSO). NMR spectra were processed using Mestrec 5.3.0 and 6.0.1. ¹³C NMR peaks that are bracketed are two rotomers of the same carbon. Mass spectra (LCMS) were obtained using an Agilent 1100/6110 HPLC system equipped with a Thompson ODS-A, 100 A, 5μ (50×4.6 mm) column using water with 0.1% formic acid as the mobile phase A, and acetonitrile with 0.1% formic acid as the mobile phase B. The gradient was 20-100% with mobile phase B over 2.5 min then held at 100% for 2.5 mins. The flow rate was 1 mL/min. For more hydrophobic compounds, the following gradient was used, denoted as Method 1: 40-95% over 0.5 min, hold at 95% for 8.5 min, then return to 40% over 2 min, with a flow rate of 1 mL/min. Final compounds were checked for purity using Method 2: 5% for 1 min, 5-95% over 9 min, then hold at 95% for 5 min, with a flow rate of 1 mL/min. Enantiomeric excess was determined by integration of peaks that were separated on a Chiralpak AD-H, 250×4.6 mm column, 5 μm particle size. Flow rate of 1 mL/min and an isocratic mobile phase. Unless otherwise indicated, the chiral data provided uses this method. Alternatively, chiral separations were performed under the following conditions, denoted as Chiral Method 1: Chiralpak AY-H, 250×4.6 mm column, 5 μm particle size. Flow rate of 1 mL/min and an isocratic mobile phase. Chiral Method 2: Chiralcel OZ-3, 250×4.6, 3 μm particle size at a flow rate of 0.75 ml/min. The pyridine, dichloromethane (DCM), tetrahydrofuran (THF), and toluene used in the procedures were from Aldrich Sure-Seal bottles kept under nitrogen (N₂). All reactions were stirred magnetically and temperatures are external reaction temperatures. Chromatographies were carried out using a Combiflash Rf flash purification system (Teledyne Isco) equipped with Redisep (Teledyne Isco) silica gel (SiO₂) columns. Preparative HPLC purifications were done on Varian ProStar/PrepStar system using water containing 0.05% trifluoroacetic acid as mobile phase A, and acetonitrile with 0.05% trifluoroacetic acid as mobile phase B. The gradient was 10-80% with mobile phase B over 12 min, hold at 80% for 2 min, and then return to 10% over 2 min with flow rate of 22 mL/min. Other methods similar to this may have been employed. Fractions were collected using a Varian Prostar fraction collector and were evaporated using a Savant SpeedVac Plus vacuum pump. Microwave heating was performed using a Biotage Initiator microwave reactor equipped with Biotage microwave vessels. The following abbreviations are used: ethanol (EtOH), carbonyldiimidazole (CDI), isopropanol (IPA), and 4-dimethylaminopyridine (DMAP).

### Example 1

### Synthesis of Compound No. 1

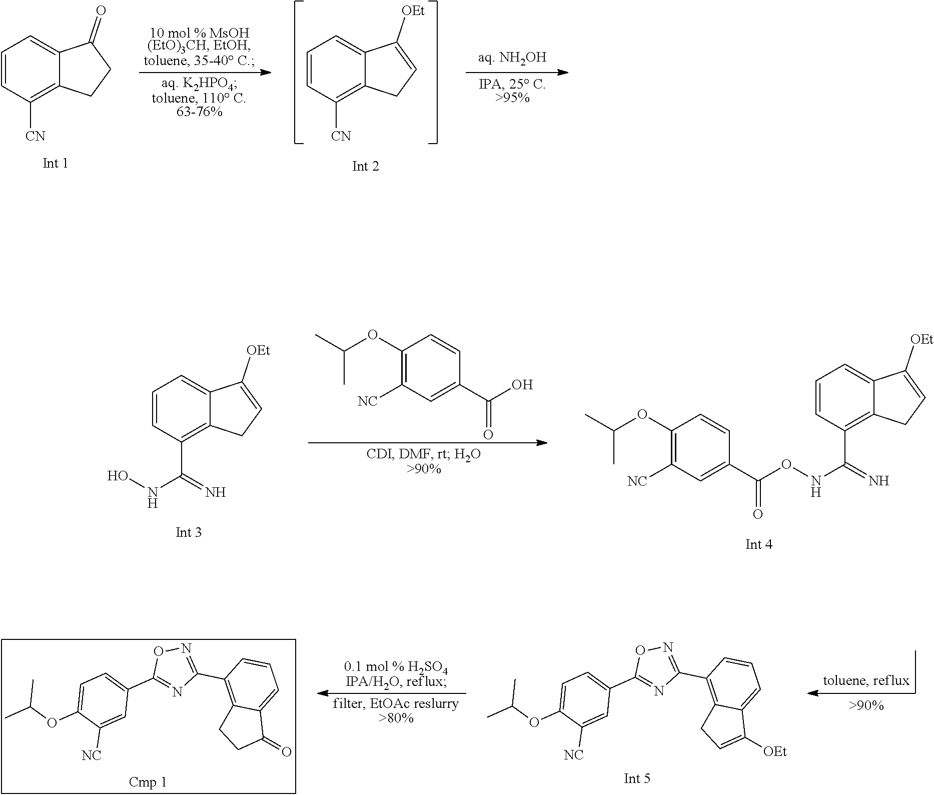

Step 1—Synthesis of 3-ethoxy-1H-indene-7-carbonitrile (Int 2):

A stirred mixture of 1-oxo-2,3-dihydro-1H-indene-4-carbonitrile (Int 1) (20.0 g, 98 wt %, 18.6 assay g, 124.8 mmol) in abs EtOH (20 mL), triethylorthoformate (80 mL, 481 mmol) and methanesulfonic acid (0.88 mL, 12.5 mmol) in toluene (80 mL) was heated at 43-47° C. After 1 h, GC analysis showed orthoformate consumed and 12.8 area % of Int 1 remaining. A further charge of triethylorthoformate (20 mL, 120.2 mmol) was made and after 45 min GC analysis showed 1.5 area % Int 1. The batch was cooled to ambient temperature and then poured into 1 M aq. K₂HPO₄ (200 mL) with vigorous stirring while maintaining a quench temperature <15° C. The two-phase mixture was vigorously stirred for 10 min. The phases were separated and the aqueous phase (pH 11) was back extracted with toluene (100 mL). The organic phases were combined and distilled at atmospheric pressure to remove 340 mL distillate. Toluene was added (500 mL) and distilled at atmospheric pressure to remove 500 mL distillate. Total distillation time 3 h, temperature range 80-120° C. At this point the batch was stored overnight at <5° C. Excess orthoformate was removed by chasing with ethyl acetate (100 mL) under reduced pressure until distillation stopped. Another volume of ethyl acetate (100 mL) was added and then concentrated under reduced pressure until distillation stopped. A third volume of ethyl acetate (100 mL) was added and then concentrated under reduced pressure until distillation stopped, after which GC analysis confirmed no orthoformate remaining. The crude was then stirred at 110° C. for 1 h, to convert the intermediate ketal to 3-ethoxy-1H-indene-7-carbonitrile (Int 2). Upon cooling, the crude (mobile oil, 21.34 g) was assayed for Int 2 by ¹H NMR employing mesitylene as an internal standard. The oil assayed at 78.1 wt % product=16.73 assay g, 90.0 mmol=72.1% assay yield. The crude oil was then purified by filtration through a silica gel plug eluting with 15% EtOAc/hexane. The pure fractions were combined and utilized for the next step. ¹H NMR (400 MHz, d₆-DMSO) δ 7.78 (d, J=8.4, 1H), 7.63 (m, 1H), 7.49 (m, 1H), 5.60 (m, 1H), 1.38 (t, J=6.8 Hz, 1H), 1.19 (t, J=6.8 Hz, 1H); LRMS: calcd for C₁₂H₁₂NO⁺ [M+H]: 186.2; Found: 186.2.

### Step 2—Synthesis of Int 3:

An EtOAc/hexane solution (650 mL) of 3-ethoxy-1H-indene-7-carbonitrile (Int 2) is concentrated under reduced pressure to ˜17 mL and isopropyl alcohol (IPA, 40 mL) was added. The solution was concentrated to ˜17 mL, and a second volume of IPA (34 mL) was added. To the stirred solution was added aqueous hydroxylamine (50%, 30 mL, 455 mmol). The batch was then warmed at 35-40° C. for 5 h, and then stirred at ambient temperature overnight. The batch was cooled to 0° C., seeded (50 mg), and stirred for 30 min for a seed bed to develop. Water (250 mL) was then added dropwise over ˜1.5 h. The batch was stirred for 1 h at 0-20° C. The product was isolated by filtration, cake-washed with water (100 mL) and dried on the filter under vacuum and a nitrogen atmosphere, to afford 3-ethoxy-N-hydroxy-1H-indene-7-carboximidamide (Int 3) (20.8 g, 90% yield). ¹H NMR (400 MHz, d₆-DMSO) δ 9.61 (s, 1H), 7.43 (m, 1H), 7.32 (m, 2H), 5.77 (s, 1H), 5.41 (s, 1H), 4.08 (q, J=6.8 Hz, 2H), 3.45 (s, 2H), 1.39 (t, J=6.8 Hz, 3H); LRMS: calcd for C₁₂H₁₅N₂O₂⁺ [M+H]: 219.2; Found: 219.1.

Step 3—Synthesis of N-((3-cyano-4-isopropoxybenzoyl)oxy)-3-ethoxy-1H-indene-7-carboximidamide (Int 4):

A mixture of CDI (16.64 g, 102.6 mmol) and 3-cyano-4-isopropoxyl benzoic acid (21.06 g 102.6 mmol) in DMF (83 mL) was stirred at 20° C. for 1 h. A solution of 3-ethoxy-N-hydroxy-1H-indene-7-carboximidamide (Int 3) (20.8 g, 93.3 mmol) in DMF (40 mL) was added through an addition funnel over ˜5 min. After ˜30 min the batch became viscous and a further volume of DMF (40 mL) was added to aid stirring. At this point HPLC assay indicated that the reaction was complete. The resulting slurry was diluted with water (1.5 L), cooled to 0° C., and isolated by filtration. The filter cake was washed with water (1.5 L) and the product dried on the filter under nitrogen flow to afford N-((3-cyano-4-isopropoxybenzoyl)oxy)-3-ethoxy-1H-indene-7-carboximidamide (Int 4) as an off white solid (34.8 g, 90% yield). ¹H NMR (400 MHz, d₆-DMSO) δ 8.70 (s, 1H), 8.33 (d, J=6.8 Hz, 1H), 7.45 (m, 4H), 7.10 (m, 2H), 5.49 (s, 1H), 4.94 (m, 1H), 4.10 (q, J=6.8 Hz, 2H), 3.55 (s, 2H), 1.38 (m, 9H); LRMS: calcd for C₂₃H₂₄N₃O₄⁺ [M+H]: 406.4; Found: 406.2.

Step 4—Synthesis of 5-(3-(3-ethoxy-1H-inden-7-yl)-1,2,4-oxadiazol-5-yl)-2-isopropoxybenzonitrile (Int 5)

N-((3-Cyano-4-isopropoxybenzoyl)oxy)-3-ethoxy-1H-indene-7-carboximidamide (Int 4) (34.8 g, 83.97 mmol) was suspended in toluene (590 mL) and heated to reflux with a Dean-Stark apparatus for 18 h. ˜2 mL were collected (theory 1.5 mL). The batch was cooled to ambient temperature, filtered through Celite, and concentrated under vacuum. The crude solid 5-(3-(3-ethoxy-1H-inden-7-yl)-1,2,4-oxadiazol-5-yl)-2-isopropoxybenzonitrile (Int 5) (30 g, 90% yield) is taken as is to the next step. LRMS: calcd for C₂₃H₂₂N₃O₃⁺ [M+H]: 388.4; Found: 388.3.

Step 5—Synthesis 2-isopropoxy-5-(3-(1-oxo-2,3-dihydro-1H-inden-4-yl)-1,2,4-oxadiazol-5-yl)benzonitrile (Cpd. No. 1):

Int 5 (30 g, 75.57 mmol) is suspended in 4:1 IPA/H₂O (300 mL). Catalytic H₂O₄ (0.1 mL, 0.19 mmol) is added, and the resulting mixture is heated to reflux for 12 h. The slurry is cooled to ambient temperature and stirred for 1 h. The product is isolated by filtration and washed with 4:1 IPA/H₂O (100 mL). After drying on the filter for 1 h under vacuum, the wet cake is charged back to the reactor and suspended in EtOAc (300 mL). The mixture is heated to reflux for 3 h, then cooled to ambient temperature and stirred for 1 h. The slurry is filtered, washed with EtOAc (100 mL), and dried on the filter under nitrogen to afford 2-isopropoxy-5-(3-(1-oxo-2,3-dihydro-1H-inden-4-yl)-1,2,4-oxadiazol-5-yl)benzonitrile (Cpd. No. 1) (22 g, 80% yield) as an off-white solid. ¹H NMR (400 MHz, d₆-DMSO) δ 8.55 (d, J=2.0 Hz, 1H), 8.44 (m, 2H), 7.88 (d, J=7.6 Hz, 1H), 7.69 (t, J=7.6 Hz, 1H), 7.57 (d, J=9.2 Hz, 1H), 4.99 (h, J=12.4 Hz, 1H), 3.46 (dd, J₁=5.6, J₂=11.2 Hz, 2H), 2.76 (dd, J₁=5.6, J₂=11.2 Hz, 2H), 1.45 (d, J=12.4 Hz, 6H); ¹³C NMR (100 MHz, d₆-DMSO) δ 205.9, 173.4, 167.4, 162.6, 154.2, 138.1, 134.7, 134.2, 133.9, 128.2, 125.9, 124.5, 115.8, 115.3, 114.9, 102.5, 72.6, 35.9, 27.3, 21.5; LRMS: calcd for C₂₁H₁₈N₃O₃⁺ [M+H]: 360.1; Found: 360.2; C,H,N Analysis: Found: % C: 70.25, % H: 4.69; % N: 11.71; Theory: % C: 70.18; % H: 4.77; % N: 11.69.

### Example 2

### Biological Activity of Compound 1

S1P_{1R} is a GPCR that couples exclusively to the inhibitory G protein (Gαi), decreasing adenylate cyclase activity and lowering cyclic adenosine monophosphate (cAMP levels) in the cell. The potency of Compound 1 was tested against the S1P1 receptor (S1P_{1R}) and related family members. The potency and S1P receptor selectivity were measured using two independent in vitro assays, [³⁵S]guanosine-5′-O-(3-thio)triphosphate ([³⁵S]GTPγS) and β-lactamase gene expression and activity. The GTPγS binding to membranes prepared from stable mammalian cells lines expressing recombinant human S1P receptors 1-5 (S1P_{1R}-S1P_{5R}) was used to pharmacologically characterize ligand interaction with the S1P receptor family members. Membranes were incubated at room temperature (RT) with test compound (10-point serial half-log dose-range with the highest concentration of 300 nM-10 μM depending upon the potency of each compound) and wheat germ agglutinin-coated scintillation proximity assay beads on a plate shaker for 30 minutes before the addition of [³⁵S]GTPγS and an additional incubation of 40 minutes at RT. Membrane-bead complexes were then pelleted by centrifugation before the bound radioactivity was quantified with a MicroBeta2® microplate scintillation counter. The radioactive counts relative to DMSO vehicle were calculated and the half maximal effective concentration (EC₅₀) was determined using non-linear regression.

Up-regulation of β-lactamase gene expression and activity were used as a whole-cell functional readout of S1P receptor activation. Stable mammalian cells lines expressing recombinant human S1P1R-S1P5R designed to couple to the gene expression of β-lactamase were pre-seeded into 384 well microplates before incubation with test compound (10-point serial 1:4 dose range with the highest concentration of 100 nM-10 μM depending upon the potency of the compound) for 4-5 hours at 37° C. in a tissue culture incubator. The respective β-lactamase activity was then determined using a fluorescence resonance energy transfer (FRET)-based β-lactamase fluorescent substrate (LiveBLAzer™-FRET B/G Loading Kit CCF4-AM) according to manufacturer's instructions, and incubated with cells for 2 hours at RT. Plates were read using a SpectraMax M5 Multi-Mode microplate reader and the relative response to DMSO vehicle calculated for the EC₅₀ determination using non-linear regression.

Compound 1 is a potent and selective S1P_{1R} and S1P_{5R} agonist in both β-lactamase activity and GTPγS binding assays, having an EC₅₀ of 2.1 nM for GTPγS binding and 0.04 nM for β-lactamase activity. Compound 1 is also active at S1P_{R5} with EC₅₀ values of 17.9 and 6.9 nM for GTPγS binding and β-lactamase activity, respectively. Compound 1 has minimal activity on S1P₂₋₄ receptors, showing greater than 1000-fold selectivity for S1P_{1R} over S1P_{2R}, S1P_{3R} and S1P_{4R} using the [³⁵S]GTPγS binding activity readout, and greater than 200-fold selectivity for S1P_{5R} over S1P_{2R}, S1P_{3R} and S1P_{4R} using both assay readouts (see Table 1).

TABLE 1
Binding Affinity for the Sphingosine-1-Phosphate Receptors
Using the GTPγS and β-Lactamase Assays

| | EC₅₀ (nM) | | | | | | | | | |
|---|---|---|---|---|---|---|---|---|---|---|
| | S1P_{1R} | | S1P_{2R} | | S1P_{3R} | | S1P_{4R} | | S1P_{5R} | |
| | | β- | | β- | | β- | | β- | | β- |
| Analyte | GTPγS | Lactamase | GTPγS | Lactamase | GTPγS | Lactamase | GTPγS | Lactamase | GTPγS | Lactamase |
| Cmp 1 | 2.1 | 0.04 | >10000 | Not tested | >5000 | >10000 | >10000 | >10000 | 17.9 | 6.9 |

### Plasma Sample Analysis

Plasma samples (50 μL) were aliquoted into a 96 deep-well polypropylene plate (2 mL/well) and 5 μL of dimethyl sulfoxide (DMSO) was added. Samples expected to be above the upper limit of quantitation (ULOQ) were diluted with plasma. Standard curves were prepared by mixing 5 μL of the test compound in DMSO at 10 times the concentration which was added to 50 μL of plasma in a 96 deep-well polypropylene plate. For example, for a 0.3 μM standard, the 10×DMSO concentration of the test compound was 3 μM. Standard curves were prepared using analytical standards of Compound 1. Protein was precipitated from study samples and standards by the addition of 150 μL of acetonitrile. The plate was vortexed to ensure complete precipitation of protein. The precipitated protein was pelleted by centrifugation at 4,000 rpm for 10 min at 20° C. and the clear supernatant was transferred to a clean 96-well plate and centrifuged again under the same conditions to pellet any transferred solid material.

### Chromatography

Samples (7 μL) were introduced to a Shimadzu HPLC (LC-20ADXR) with a SIL-30ACMP autosampler (Shimadzu). The mobile phase was a gradient using 0.1% formic acid in water and 0.1% formic acid in acetonitrile. The column used was a Phenomenex Kinetic C18 100 A 2.6μ30×3 mm (PN 946975-906). The CTO-20AC column oven (Shimadzu) was set at 40° C. Prepared samples were usually analyzed in order of lowest to highest concentration for standards and reverse order of time-points for study samples. Study samples were bracketed by full standard curves. Typically at least 6 standards were used for quantitation with a percent accuracy of +/−15% for all standards except at the lower level of quantitation (LLOQ) where a percent accuracy of +/−20% was allowed.

### Mass Spectrometry

An ABSciex Instruments Triple Quad 5500 Mass Spectrometer (Analyst 1.6.3) was used for detection in the MRM mode. Ionization was achieved by positive or negative electrospray with a source temperature of 600° C. Negative and positive mode analysis was performed in the same injection. Standard samples were usually analyzed in order of lowest to highest concentration. Typically at least six standards were used for quantification with a percent accuracy of +/−15% for all standards except at the LLOQ where a percent accuracy of +/−20% was allowable. Study samples were analyzed in reverse chronological order bracketed with bracketing standard curves.

### Determination of Absolute Oral Bioavailability in Rats

Pharmacokinetic studies are conducted in non-fasted male Sprague-Dawely rats (Simonsen Laboratories or Harlan Laboratories). Rats are housed in an ALAAC accredited facility and the research approved by the facilities Institutional Animal Care and Use Committee (IACUC). The animals are acclimated to the laboratory for at least 48 h prior to initiation of experiments.

Compound 1 was formulated in 5% DMSO/5% Tween20 and 90% purified water (intravenous infusion) or 5% DMSO/5% Tween20 and 90% 0.1N HCL (oral gavage). The concentration of the dosing solutions is verified by HPLC-UV. For intravenous dosing, compounds were administered by an infusion pump into the jugular vein over one minute to manually restrained animals (n=4 rats/compound). Oral dosing is by gavage using a standard stainless steel gavage needle (n=2-4 rats/compound). For both routes of administration, blood is collected at eight time-points after dosing with the final sample drawn 24 h post dose. Aliquots of the blood samples are transferred to polypropylene 96-well plate and frozen at −20° C. until analysis.

After thawing the blood samples at room temperature, 5 μL of DMSO is added to each well. Proteins are precipitated by adding 150 μL acetonitrile containing 200 nM internal standard (4-hydroxy-3-(alpha-iminobenzyl)-1-methyl-6-phenylpyrindin-2-(1H)-one) and 0.1% formic acid. Plates are mixed for 1 min on a plate shaker to facilitate protein precipitation and then centrifuged at 3,000 rpm for 10 min to pellet protein. The supernatant is transferred to a clean plate and centrifuged at 3,000 rpm for 10 min to pellet any remaining solid material prior to LC/MS/MS analysis. Calibration curve standards are prepared by spiking 5 μL compound stock in DMSO into freshly collected EDTA rat blood. An eight point standard curve spanning a range of 5 nM to 10,000 nM is included with each bio-analytical run. The standards are processed identically to the rat pharmacokinetic samples.

Concentrations in the rat pharmacokinetic samples are determined using a standardized HPLC-LC/MS/MS method relative to the eight point standard curve. The system consists of a Leap CTC Pal injector, Agilent 1200 HPLC with binary pump coupled with an Applied Biosystems 3200 QTrap. Compounds are chromatographed on a Phenomenex Synergy Fusion RP 20×2 mm 2 um Mercury Cartridge with Security Guard. A gradient method is used with mobile phase A consisting of 0.1% formic acid in water and mobile phase B consisting of 0.1% formic acid in acetonitrile at flow rates varying from 0.7 to 0.8 mL/min. Ions are generated in positive ionization mode using an electrospray ionization (ESI) interface. Multiple reaction monitoring (MRM) methods are developed specific to each compound. The heated nebulizer is set at 325° C. with a nebulizer current of 4.8 μA. Collision energies are used to generate daughter ions ranged between 29 and 39 V. Peak area ratios are obtained from MRM of the mass transitions specific for each compound used for quantification. The limit of quantification of the method is typically 5 nM. Data are collected and analyzed using Analyst software version 1.4.2.

Blood concentration versus time data are analyzed using non-compartmental methods (WinNonlin version 5.2; model 200 for oral dosing and model 202 for intravenous infusion). Absolute oral bioavailability (%) is calculated using the following expression: (Oral AUC×IV Dose)/(IV AUC×Oral Dose)×100.

### Compound 1 Single Dose Pharmacokinetics in the Rat

Compound 1 was administered in a formulation of 10% volume/volume (v/v) DMSO and 5% v/v Tween 20 in water by IV administration (10 mL/kg) or 0.5% weight/volume (w/v) CMC in water by oral gavage (10 mL/kg). Animals received 0.2 mg/kg as the IV dose or 2 mg/kg/day orally as a single administration. Blood samples were collected at 1, 5, and 30 minutes, 2, 4, 8, 10, 24, 24, 48, and 72 hours after IV dosing and 1, 2, 3, 4, 6, 8, 10, 12, 24, 48, and 72 hours following oral dosing. Blood was collected into tubes containing the anti-coagulant K₂EDTA and processed to plasma. Plasma concentrations for each analyte were determined using a qualified LC-MS/MS method. The PK parameters are summarized in Table 2 below.

TABLE 2
Mean Pharmacokinetic Parameters After a Single
Administration of Compound 1

| Dose | | | | AUC₀₋₂₄ | | | |
|---|---|---|---|---|---|---|---|
| (mg/kg); | | tₘₐₓ | Cₘₐₓ | (hr* | t½ | Vd/F | |
| Route | Analyte | (hr) | (nmol/L) | nmol/L) | (hr) | (L/kg) | % F |
| 0.2 | Cmp 1 | 0.02 | 743 | 386 | 1.62 | DNS | NA |
| IV | | | | | | | |
| 2.0 | Cmp 1 | 3.00 | 14.8 | 95.4 | 3.73 | 292 | 2.5 |
| Oral | | | | | | | |

Abbreviations:
DNS = data not sufficient;
NA = not applicable.

### Assessment of Circulating Lymphocyte Counts

Whole blood samples were collected from acclimated, conscious male Sprague Dawley rats in the fed state using percutaneous jugular vein puncture at pre-dose and 3, 6 and 24 hours after single oral dose administration of vehicle or test compound. Blood samples were collected into K₂EDTA tubes, thoroughly mixed and stored on wet ice until analyzed. Circulating lymphocyte counts were assessed by two methods from individual aliquots of the same samples. Total lymphocyte counts were determined in a standard hematology panel on a calibrated analyzer (Test #416; Horiba Medical ABX Pentra XL 80) and by in-house flow cytometry using fluorescent-activated cell sorting (FACS) analysis to determine the number of CD4+, CD8+ and B220+ lymphocytes (ThermoFisher Attune NxT flow cytometer). Data are expressed as follows for absolute lymphocyte counts (hematology analyzer), the individual lymphocyte subtypes (CD4+, CD8+ and B220+) and the sum of the individual lymphocyte subtypes (flow cytometry).
Number of lymphocytes per μL whole blood.
Percentage change relative to each group's pre-dose baseline at each time point

### Flow Cytometry Procedures

Whole blood samples (K₂EDTA) were analyzed on the flow cytometer (ThermoFisher Attune NxT) using rat antibodies, as detailed below. The baseline and 3 hour post-dose samples were analyzed on the same day as collection. The 6 hour post-dose samples were analyzed after overnight storage at 4° C. (on wet ice in a 2-8° C. fridge) and on the same day as the 24 hour post-dose samples were collected and analyzed.

### Pharmacodymanic Effects

The oral dose level of 2 mg/kg of Compound 1 administered to the animals was intended to induce a significant reduction (˜≥60%) in the number of circulating lymphocyte. Following acclimation, blood samples were serially collected from the jugular vein of conscious Sprague Dawley rats prior to dosing and 3 hours, 6 and 24 hours after administration. The absolute number of circulating lymphocytes was quantified in whole blood samples using a hematology analyzer and the CD4⁺, CD8⁺ and B220⁺ lymphocyte subtypes by flow cytometry. The plasma concentrations for each compound were quantified with liquid chromatography, tandem mass spectrometry.

Oral administration of Compound 1 significantly reduced total circulating lymphocyte counts (total number of CD4⁺, CD8⁺ and B220⁺) by 52% relative to pre-dose values (see Figure). The effects on individual lymphocyte subtypes were similar with CD4⁺, CD8⁺ and B220⁺ lymphocyte populations reduced by 68%, 62% and 35%, respectively. Furthermore, measurement in a hematology analyzer also gave similar results (44% fewer lymphocytes relative to pre-dose values). In general, the maximum effect was observed by six hours after administration. Compound 1 was without significant effect at the 24 hour time-point. Lymphocyte counts reflected the plasma concentrations of Compound 1 after administration, demonstrating a clear pharmacokinetic/pharmacodynamic (PK/PD) relationship.

### Clinical PK of Compound 1

Compound 1 pharmacokinetic (PK) parameters were derived from a Phase 1 study in which approximately 24 eligible subjects with relapsing remitting multiple sclerosis (RMS) received an initial 7-day dose escalation of ozanimod HCl (0.25 mg PO QD on Day 1 to Day 4 and 0.5 mg PO QD on Day 5 to Day 7) followed by either 0.5 mg PO QD (Group 1, n=12) or 1 mg PO QD (Group 2, n=12) until approximately Day 85±5 days. Serial blood PK samples were collected on Days 1 and the last day of dosing. As summarized in Table 3, PK parameters for ozanimod and its metabolites including Compound 1 were estimated using non-compartmental analysis and actual PK collection times.

TABLE 3
Summary of Clinical PK Properties of Ozanimod and Active Metabolites

| | Ozanimod | Compound 1 |
|---|---|---|
| Median Tmax | 6-8 hours | 6-10 hours |
| Mean t1/2 (at steady state) | 15-23 hours | ca. 10-13 days |
| Peak-to-trough ratio (at | 2-fold | 1.3- to 1.6-fold |
| steady state) | | |
| % of total agonist | Ozanimod: 3% to 6% | 85% to 93% |
| exposure* at steady state | Other metabolites 5% to 9% | |

*Total agonist = sum of ozanimod and active metabolites

Comparative selectivity for Ozanimod and Compound 1 with regard to S1P₁-S1P₅ are shown in Table 4, with the agonist values (EC₅₀) reported in nM.

TABLE 4
Comparative Selectivity

| Distribution | | S1P₁ | S1P₂ | S1P₃ | S1P₄ | S1P₅ |
|---|---|---|---|---|---|---|
| EC₅₀ | Ozanimod | 1.2 | >10,000 | >5,000 | 2038 | 5.1 |
| | Compound 1 | 2.1 | >10,000 | >5,000 | >10,000 | 17.9 |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. U.S. Provisional Application 62/839,495, filed Apr. 26, 2019 is incorporated herein by reference, in its entirety.

We claim:

1. An isolated or purified form of a compound having the following structure (Compound 1): 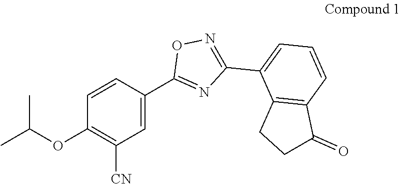 [SMILES: CC(C)Oc1ccc(-c2nc(-c3cccc4c3CCC4=O)no2)cc1C#N]
or a pharmaceutically acceptable salt, homolog, hydrate or solvate thereof.

2. The isolated or purified form of the compound of claim 1, wherein Compound 1 is present at a purity in excess of 90% (w/w).

3. The isolated or purified form of the compound of claim 1, wherein Compound 1 is present at a purity in excess of 95% (w/w).

4. The isolated or purified form of the compound of claim 1, wherein Compound 1 is present at a purity in excess of 98% (w/w).

5. The isolated or purified form of the compound of claim 1, wherein Compound 1 is present at a purity in excess of 98% (w/w).

6. The isolated or purified form of the compound of claim 1, wherein Compound 1 is present at a purity in excess of 99% (w/w).

7. A pharmaceutical composition comprising a compound having the following structure (Compound 1): 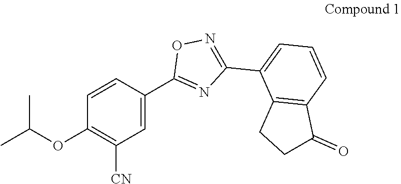 [SMILES: CC(C)Oc1ccc(-c2nc(-c3cccc4c3CCC4=O)no2)cc1C#N]
or a pharmaceutically acceptable salt, homolog, hydrate or solvate thereof, in combination with a pharmaceutically acceptable carrier or diluent.

8. The pharmaceutical composition of claim 7, wherein the composition is in a form suitable for oral administration.

9. The pharmaceutical composition of claim 7, wherein the composition is in a form suitable for intravenous administration.
